(19) United States
(12) Patent Application Publication
(10) Pub. No.: US 2009/0125032 A1
(43) Pub. Date: May 14, 2009

(54) Rod removal instrument

(76) Inventors: Robert C. Gutierrez, Huntington Beach, CA (US); Stanley Kyle Hayes, Mission Viejo, CA (US)

Correspondence Address: RIMAS LUKAS;VERTIFLEX, INC., 1351 CALLE AVANZADO, SAN CLEMENTE, CA, 92673, US

(21) Appl. No.: 12/270,505

(22) Filed: Nov. 13, 2008

Related U.S. Application Data
(60) Provisional application No. 61/002,985, filed on Nov. 14, 2007.

(51) Int. Cl. A61B 17/58; A61B 19/00

(52) U.S. Cl. 606/99; 128/898

(57) ABSTRACT

A rod removal instrument for removing a rod from the seat of a vertebral anchor is disclosed. The rod removal instrument comprises an outer shaft connected to an inner shaft. The outer shaft has a rod receiving portion at its distal end. When placed in juxtaposition with the rod, the rod removal instrument is rotated to hook the rod into the rod receiving portion. The inner shaft is then moved distally with respect to the outer shaft to dislodge the rod from the seat and capture it in the rod removal instrument for removal from the surgical site.

### CROSS-REFERENCE TO RELATED APPLICATIONS

This application claims the benefit of and is a continuation-in-part of U.S. Provisional Patent Application Ser. No. 61/002,985 entitled “Rod removal instrument” filed on Nov. 14, 2007 which is incorporated herein by reference in its entirety.

### FIELD

The present invention generally relates to medical devices for the spine. In particular, the present invention relates to an instrument for removing a spinal rod from a spinal bone anchor.

### BACKGROUND

Damage to the spine as a result of advancing age, disease, and injury, has been treated in many instances by fixation or stabilization of vertebrae. Conventional methods of spinal fixation utilize a rigid spinal fixation device to support an injured spinal vertebra relative to an adjacent vertebra and prevent movement of the injured vertebra relative to an adjacent vertebra. One spinal fixation system includes bone anchor members 10 for fixing to a series of vertebrae of the spine and at least one rigid or semi-rigid link element 12 designed to interconnect the anchor members 10 as shown in FIGS. 1 and 2. Typically, the rigid link element is a rod 12. An example of one type of rod 12 is shown in FIG. 3 wherein one end of the rod is partially spherical in shape and includes coupling constructs 20 in the form of small pins for attachment to anchor members 10. The other end of the rod 12 of FIG. 3 is typically oriented to engage the lower adjacent vertebra and may or may not have a spherical end and coupling constructs.

Still referencing FIGS. 1 and 2, an anchor member 10 is generally a bone screw 14 polyaxially coupled to a seat 18. The seat 18 typically includes a substantially U-shaped channel 22 for receiving either end of the rod 12 of FIG. 3. A closure mechanism (not shown) closes the open end of the seat 18 to lock the rod 12 inside.

The screw 14 is configured to be inserted into the pedicle of a vertebra 16 to a predetermined depth and angle. One end of the rigid link element 12 is connected to an anchor 10 inserted in the pedicle of an upper vertebra and the other end of the rod 12 is connected to an anchor 10 inserted in the pedicle of a lower vertebra 16 with some overhang at the caudal end. At least one of the rod ends is connected to an anchor via coupling constructs 20 which can be of the type shown in FIG. 3 or via other means. Typically two rods 12 and two pairs of anchors 10 are installed each in the manner described above as shown in FIG. 2 such that two rods are employed to fix two adjacent vertebrae 16, with one rod 12 positioned on each side of the spinous processes of adjacent vertebrae such that the adjacent vertebrae are supported and held apart in a relatively fixed position by the rods. In FIG. 1, there are shown two rods 12 and three pairs of anchors 10 to fix three adjacent vertebrae. Once the system has been assembled and fixed to a series of two or more vertebrae, it constitutes a rigid device substantially preventing the vertebrae from moving relative to one another. This rigidity enables the devices to support all or part of the stresses instead of the stresses being born by the series of damaged vertebra.

Sometimes it is necessary to remove an in-situ rod 12 for a number of reasons. It may be necessary to reposition a rod or replace a rod with a different rod. Removal of these rods 12 can be difficult given the attendant circumstances which typically involve small percutaneous or minimally invasive openings through which the rod is to be removed and obstructed views of the rod in the surgical site. Furthermore, some rods are securely attached to the seat via various coupling constructs which require additional attention for rod removal purposes. All of these circumstances compound the rod removal process and, therefore, an improved rod removal instrument is required to facilitate and expedite the procedure.

### SUMMARY

According to one aspect of the invention, a rod removal instrument for removing a rod from a spinal anchor member implanted in a patient's spine is disclosed. The rod removal instrument comprises an outer shaft having a distal end configured to at least partially encompass or cover the spine anchor member. The outer shaft includes a hook portion formed at the distal end configured to remove the rod from the spinal anchor member. In one variation, the hook portion forms a rod entryway interconnected to a rod receiving portion. Both the rod entryway and rod receiving portion are interconnected and sized to receive the rod. In another variation, the rod removal instrument further includes an inner shaft connected to the outer shaft. The inner shaft is located inside the outer shaft and configured to be longitudinally movably with respect to the outer shaft. In another variation, the inner shaft includes a notch formed at the distal end and the inner shaft is connected to the outer shaft such that the notch is displaced a distance from the rod entryway of the outer shaft. The notch is configured to conform to and to partially encompass or cover a rod. The distal end of the inner shaft is configured to retain the rod to the instrument when the inner shaft is moved distally to contact the rod. In another variation, the inner shaft is configured to dislodge the rod from the spinal anchor member when the inner shaft is moved distally to contact the rod. The rod removal instrument further includes an outer shaft handle connected to the proximal end of the outer shaft and an inner shaft handle connected to the proximal end of the inner shaft.

According to another aspect of the invention, a rod removal instrument for removing a rod connected to an anchor member implanted in a patient's spine is disclosed. The rod removal instrument comprises an elongated member having a distal end configured to percutaneously disconnect a rod from an anchor member to which the rod is connected and hold the rod for removal of the rod from the patient. In one variation, the elongated member is an outer shaft. In another variation, in addition to the outer shaft, the rod further includes an inner shaft located within the outer shaft and configured for relative sliding motion with respect to the outer shaft. In one variation, the inner shaft is configured to hold the rod against the outer shaft when the inner shaft is moved relative to the outer shaft against the rod with a rod properly positioned with respect to the rod removal instrument. In another variation, the relative sliding motion of the inner and outer shafts acts to dislodge a rod from the bone anchor. In another variation, the distal end is configured to be placed over or cover at least a portion of the anchor member. In another variation, the rod removal instrument is configured for insertion into a patient and removal of a rod from a patient through an opening in a patient that is as large as or slightly larger than the outer perimeter of the distal end. In another variation, the distal end defines a rod entryway interconnected with a rod receiving portion and configured such that the rod is insertable into the rod receiving portion through the rod entryway for removal of the rod from the anchor member. In another variation, removal instrument of claim 14 wherein rod removal instrument is configured such that a rod is insertable into the rod receiving portion after passing a portion of the rod through the rod entryway and turning the distal end relative to the rod to insert the rod into the rod receiving portion.

According to another aspect of the invention, a method for removing a rod from a spinal anchor member having a seat implanted in a patient's spine is disclosed. The method includes the step of providing a rod removal instrument comprising an outer shaft which has a rod receiving portion at the distal end. The distal end of the rod removal instrument is placed in juxtaposition with the rod to be removed. The rod is hooked into the rod receiving portion. The rod is captured inside the rod removal instrument and removed from the anchor member. In another variation, the method further includes the step of creating an opening in a patient that is as large as or slightly larger than the outer shaft and inserting the distal end of the rod removal instrument into the opening. The rod and rod removal instrument are removed from the opening. In another variation, the rod removal instrument includes an inner shaft located within the outer shaft. The inner shaft is moved with respect to the outer shaft such that the rod is captured between the inner shaft and the outer shaft and the inner shaft is held against rod. In one variation, the step of moving the inner shaft with respect to the outer shaft includes disengaging any connecting constructs of the rod from the anchor member. In another variation, the step of hooking the rod into the rod receiving portion includes turning the outer shaft relative to the rod.

### BRIEF DESCRIPTION OF THE DRAWINGS

The invention is best understood from the following detailed description when read in conjunction with the accompanying drawings.

FIG. 1 illustrates a perspective view of several bone anchors implanted into a portion of a human spine and two rods and a rod removal instrument according to the present invention.

FIG. 2 illustrates a partial perspective view of two bone anchors without closure mechanisms and a rod connected therebetween implanted in a patient's spine.

FIG. 3 illustrates a perspective view of a rod.

FIG. 4a illustrates a perspective view of a rod removal instrument according to the present invention.

FIG. 4b illustrates a front view of a rod removal instrument according to the present invention.

FIG. 5 illustrates a cross-sectional view taken along line A-A of FIG. 4b of a rod removal instrument according to the present invention.

FIG. 6a illustrates a front view of an outer shaft of a rod removal instrument according to the present invention.

FIG. 6b illustrates a side view of an outer shaft of a rod removal instrument according to the present invention.

FIG. 6c illustrates a cross-sectional view taken along line A-A of FIG. 6b of an outer shaft of a rod removal instrument according to the present invention.

FIG. 6d illustrates an end view of an outer shaft of a rod removal instrument according to the present invention.

FIG. 7a illustrates a front view of an inner shaft of a rod removal instrument according to the present invention.

FIG. 7b illustrates a cross-sectional view taken along line A-A of FIG. 7a of an inner shaft of a rod removal instrument according to the present invention.

FIG. 8a illustrates a perspective view of a rod removal instrument according to the present invention in juxtaposition with a portion of a patient's spine with a rod and two bone anchors implanted therein.

FIG. 8b illustrates a partial perspective view of a rod removal instrument according to the present invention in juxtaposition with a rod and a bone anchor implanted in a patient's spine.

FIG. 8c illustrates a perspective view of a rod removal instrument according to the present invention, two bone anchors and a rod implanted in a patient's spine.

FIG. 8d illustrates a partial perspective view of a rod removal instrument according to the present invention in juxtaposition with a rod and a bone anchor implanted in a patient's spine.

FIG. 8e illustrates a front view of a rod removal instrument according to the present invention connected to a rod in juxtaposition to a bone anchor.

FIG. 8f illustrates a front view of a rod removal instrument according to the present invention.

### DETAILED DESCRIPTION

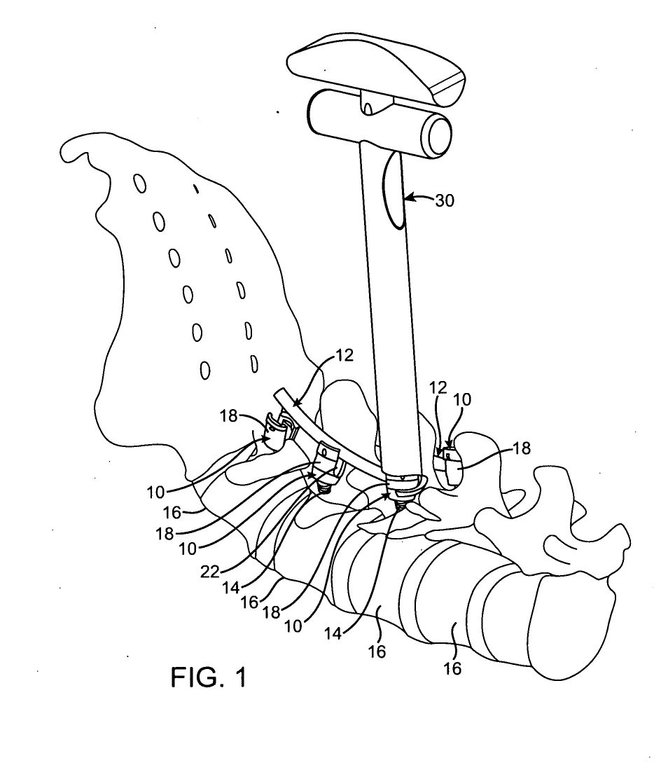

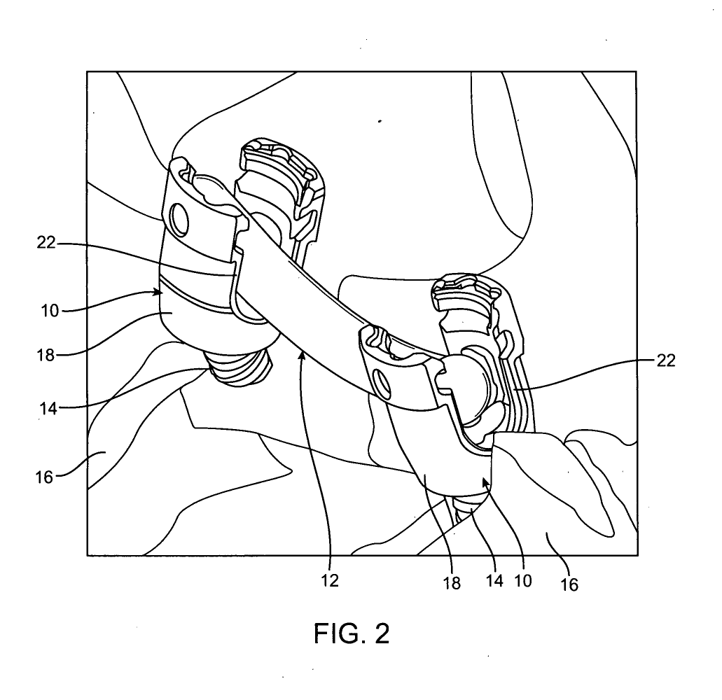

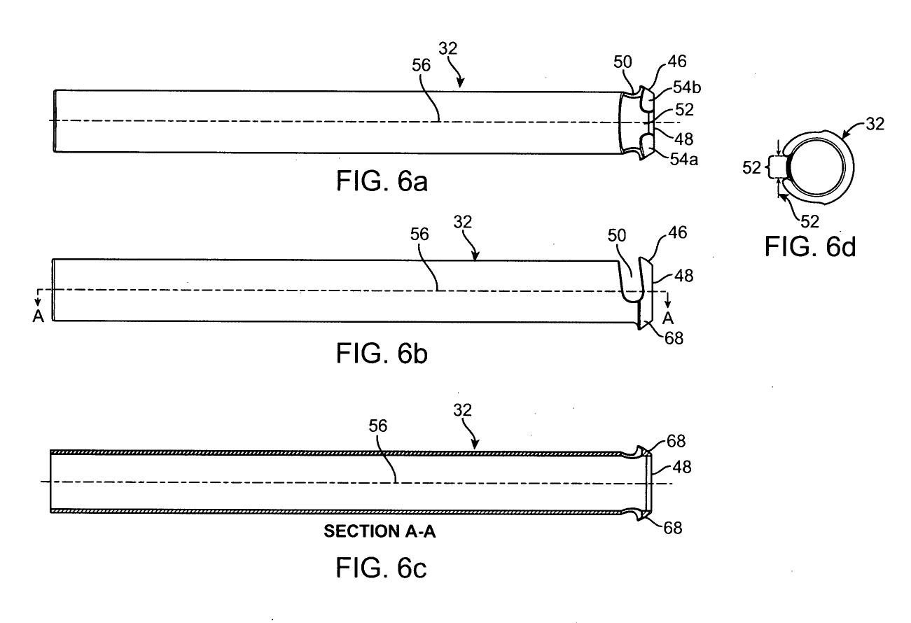

Referring now to FIGS. 1, 4a, 4b and 5 there is shown a rod removal instrument 30 according to the present invention. The rod removal instrument 30 includes an outer shaft 32 connected to an outer shaft handle 34 by a first pin 40 or other connecting means. The rod removal instrument 30 further includes an inner shaft 36 connected to an inner shaft handle 38 by a second pin 42 or other connecting means. The inner shaft 36 is disposed inside the outer shaft 32 and longitudinally movable with respect to the outer shaft 32. A spring 44 is located between the inner and outer shafts 32, 36 to bias the inner shaft 36 with respect to the outer shaft 32. Referring now to FIGS. 6a-6d, there is shown the outer shaft 32 of the rod removal instrument 30 according to the present invention. The outer shaft 32 is tubular in shape and includes a rod hook 46 at the distal end 48. The rod hook 46 forms a rod receiving portion 50 clearly seen in FIG. 6b. The rod receiving portion 50 is oriented approximately 8 degrees with respect to the transverse axis of the outer shaft 32 as shown in FIG. 6b and extends through the outer shaft 32. The rod receiving portion 50 is sized to receive a rod 12.

The rod hook 46 also forms a rod entryway 52 clearly seen in FIGS. 6a and 6d. The rod entryway 52 is interconnected with the rod receiving portion 50. The rod entryway 52 is sized to allow a rod 12 to pass therethrough. The rod hook 46 includes two substantially circumferential prongs 54a, 54b that curve around at least part of the distal circumference of the outer shaft 32 to form the gap of the rod entryway 52. The prongs 54a, 54b substantially lie in one plane generally perpendicular to the longitudinal axis 56 of the outer shaft 32. The proximal surface of the prongs 54a, 54b is slanted to match the approximately 8 degree angle of the rod receiving portion 50. The proximal surface of the prongs 54a, 54b form the distal surface of the rod receiving portion 50. The outer surface 68 of the prongs 54a, 54b is slanted at approximately 55 degrees as can be seen in FIG. 6c. The outer surface 68 of the prongs 54a, 54b is angled to prevent tissue from catching in between the rod removal instrument 30 and an anchor member 10 when the instrument 30 is inserted into the patient. For example, as the instrument 30 is inserted into the patient over an anchor member 10, the angled outer surface 68 plows tissue away from the anchor member 10. The distal end 48 of the outer shaft 32 is sized to at least partially encompass an anchor member 10. In particular, the distal end 48 of the outer shaft 32 is sized to least partially and circumferentially encompass or cover the seat 18 of the anchor member 10. The rod receiving portion 50 is configured such that when connected to a seat 18 of an anchor member 10, the prongs 54a, 54b are movable to a position underneath the rod 12 with the rod 12 being captured within the rod receiving portion 50. The use of the rod removal instrument 30 will be described in greater detail below.

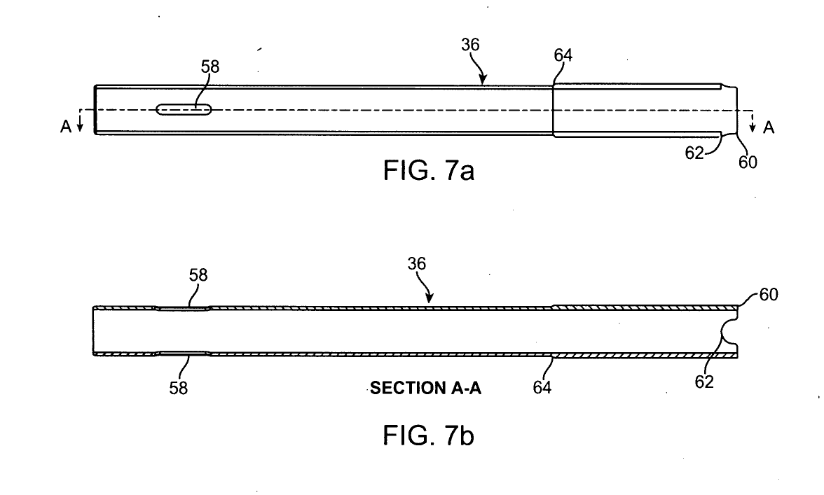

Turning now to FIGS. 7a and 7b, there is shown an inner shaft 36 according to the present invention. The inner shaft 36 is tubular in shape and is sized to fit concentrically inside the outer shaft 32. Two slots 58, located directly opposite from one another, are formed in the sidewall of the inner shaft 36 for receiving a pin and limiting longitudinal travel of the inner shaft 36 with respect to the outer shaft 32. At the distal end 60 of the inner shaft 36, a notch 62 is formed. The notch 62 is shaped and sized to at least partially encompass or cover a rod 12. As shown, the notch 62 is approximately semi-circular in shape and larger in circumference than the circumference of a rod 12. The shape of the notch 62 need not be semi-circular so long as it substantially corresponds to the shape of the rod 12 being removed. The inner shaft 36 is longer than the outer shaft 32. The inner shaft 36 further includes a shoulder 64 extending at least partially around the outer surface of the inner shaft 36.

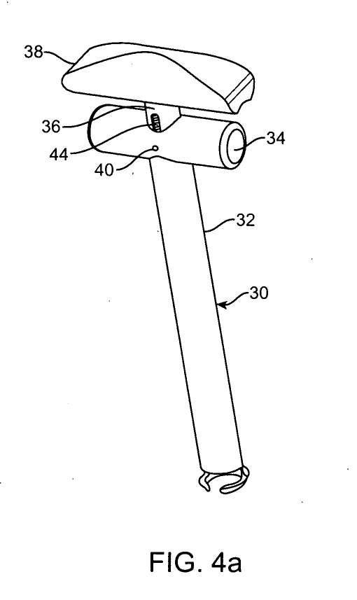

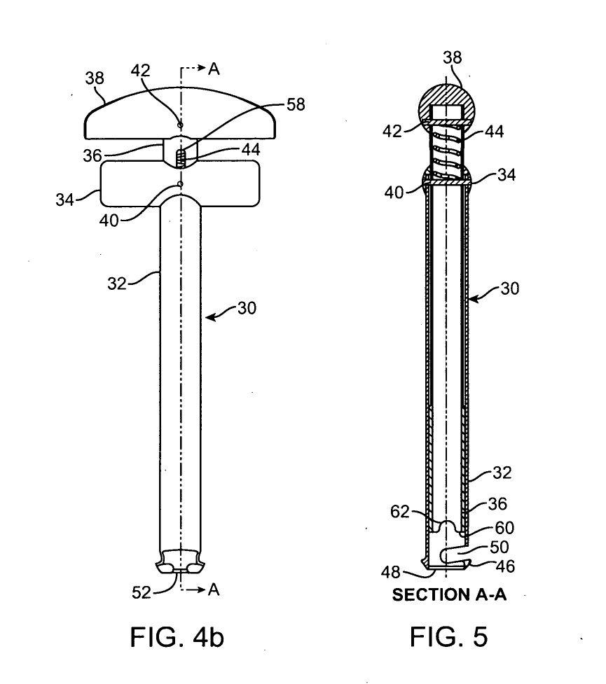

With particular reference back to FIGS. 4b and 5, the assembly of the rod removal instrument 30 will now be described. The inner shaft 36 is inserted into the larger central opening of the outer shaft 32. The outer shaft handle 34 is pass over the proximal end of the inner shaft 36 and pressed into location onto the outer shaft 32. The inner shaft handle 38 is attached to the inner shaft 36 at the proximal end of the inner shaft 36 via connecting means such as the second pin 42 or press-fit connection. The spring 44 is then inserted into the central longitudinal opening at the distal end 60 of the inner shaft 36 and moved to the proximal end. The spring 44 is compressed and the first pin 40 is passed through the outer shaft handle 34, outer shaft 32 and slot 58 of the inner shaft 36 such that a spring bias is established forcing the outer and inner shafts 32, 36 apart. With the outer and inner shafts 32, 36 biased apart by the spring 44, the distal end 60 of the inner shaft 36 clears the rod hook 46 as shown in FIG. 5. The rod removal instrument 30 is configured such that the notch 62 of the inner shaft 36 is not aligned with the rod entryway 52 but displaced a distance therefrom, preferably a distance at least equal to the diameter of a rod having a circular cross-section or equivalent width of a rod having a different cross-section. In the variation shown in the figures, the notch 62 is displaced from the rod entryway 52 by approximately 90 degrees. The inner shaft 36 is longitudinally movable with respect to the outer shaft 32 by a distance approximately equal to the length of slot 58. When the inner shaft 36 is moved distally with respect to the outer shaft 32, the spring 44 is compressed until the first pin 40 abuts the proximal end of the slot 58 at which point the distal end 60 of the inner shaft 36 is approximately in alignment with the distal end 48 of the outer shaft 32 and the rod receiving portion 50 of the outer shaft 32 is partially closed by the distal end 60 of the inner shaft 36 leaving only a circular opening sized slightly larger than the a rod 12 wherein the circular opening is formed in part by the semi-circular notch 62 and the distal surface of the rod receiving portion 50. The spring 44 biases the inner shaft 36 proximally relative to the outer shaft 32 when the handle 38 is released.

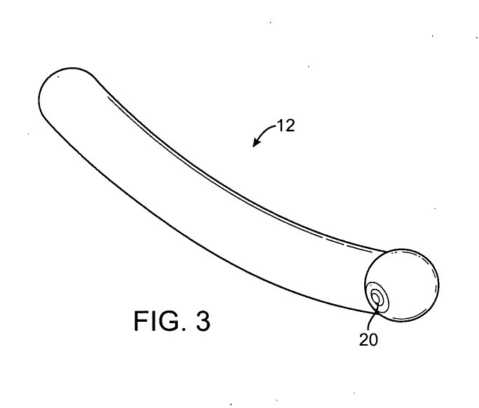

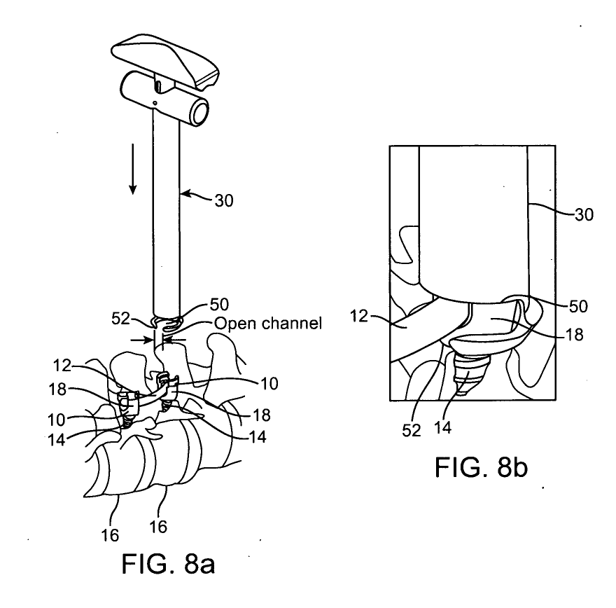

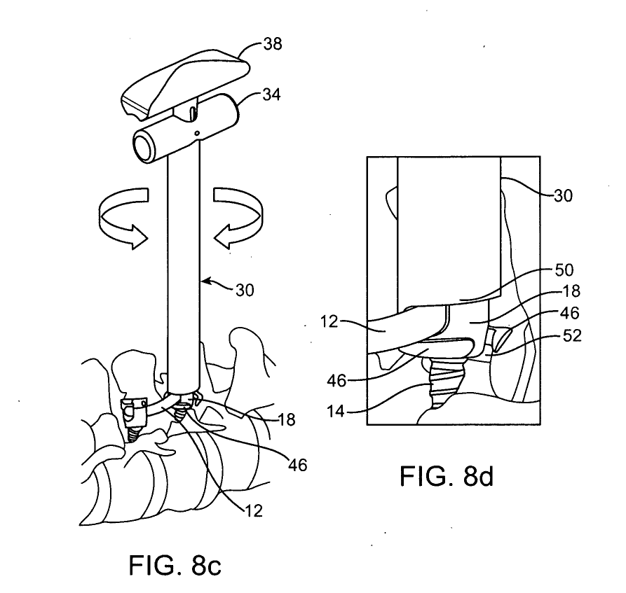

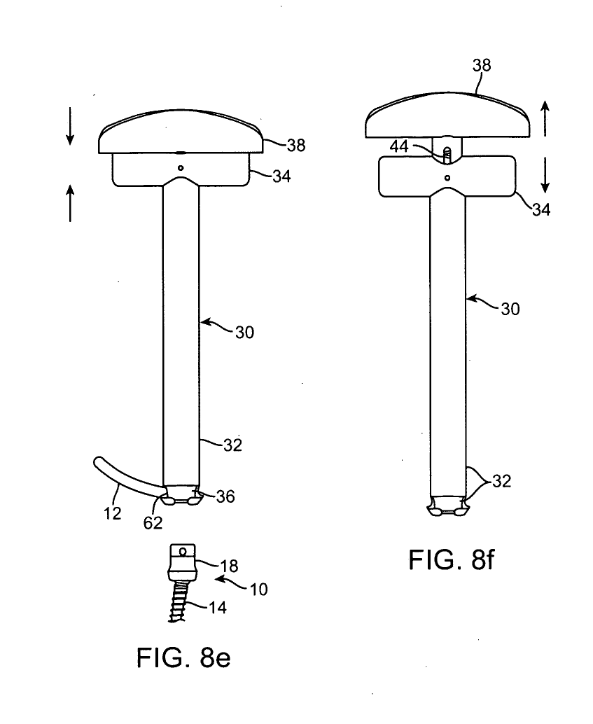

Turning now to FIGS. 8a-8f, and with particular reference first to FIG. 8a, there is shown a rod removal instrument 30 located above a spine with two anchor members 10 implanted in adjacent vertebrae 16. A rod 12 is shown located between the two anchor members 10 in FIG. 8a. Each anchor member 10 includes a screw 14 (partially visible) and a seat 18 having a U-shaped channel. In use, the rod removal instrument 30 is placed in juxtaposition with the rod 12 such that the rod entryway 52 is aligned with the rod 12 to allow entry of the rod 12 through the rod entryway 52 into the rod receiving portion 50. The rod removal instrument 30 is positioned over the seat 18 in the direction of the arrow shown in FIG. 8a. The rod 12 is passed into the rod entryway 52 until the rod 12 abuts the proximal end of the rod receiving portion 50 and the rod removal instrument 30 at least partially encompasses or covers the seat 18 as shown in FIG. 8b. Next, the rod removal instrument 30 is rotated in either a clockwise or counterclockwise direction as shown by the arrows in FIG. 8c to move one of the rod hooks 46 under the rod 12 to hook and seat the rod 12 inside the rod receiving portion 50 as shown in close-up in FIG. 8d. In the variation shown in FIGS. 8a-8f, the rod removal instrument 30 is rotated approximately 90 degrees to hook and seat the rod 12 inside the rod receiving portion 50. The outer shaft handle 34 and the inner shaft handle 38 are squeezed together as shown by the arrows in FIG. 8e. As the handles are squeezed, the relative motion of the outer and inner shafts 32, 36 results in the inner shaft 36 moving distally with respect to the outer shaft 32. The inner shaft 36 covers the rod 12 from the top and captures the rod 12 between the notch 62 and prongs 54a, 54b inside the rod receiving portion 50. As the inner shaft 36 moves distally relative to the outer shaft 32, the inner shaft 36 exerts a force to dislodge the rod 12 from the seat 18. For example, if coupling constructs 20 such as the pins 20 shown in FIG. 3 are employed, the pins are urged from their slots to remove the rod 12 from the seat 18 as shown in FIG. 8e. The hooked rod 12 is captured and lifted upwardly and removed from the patient. So long as the handles 34, 38 are compressed, the rod 12 will not drop out of the instrument 30. Once clear of the surgical site, the rod 12 can be released by releasing the handles 34, 38 which are biased apart by the spring 44 in the direction shown by the arrows in FIG. 8f. As seen in FIG. 8f, with the handles 34, 38 released, the inner shaft 36 has moved proximally inside the outer shaft 32 and out of the rod receiving portion 50 to clear the rod entryway 52 and receiving portion 50 for the next rod 12 to be removed.

The disclosed devices or any of their components can be made of any biologically adaptable or compatible materials including PEEK, PEK, PAEK, PEKEKK or other polyetherketones. Materials considered acceptable for biological implantation are well known and include, but are not limited to, stainless steel, titanium, tantalum, combination metallic alloys, various plastics, polymers, resins, ceramics, biologically absorbable materials and the like.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

We claim:

1. A rod removal instrument for removing a rod from a spinal anchor member implanted in a patient's spine; the rod removal instrument comprising:
an outer shaft having a distal end configured to at least partially encompass or cover the spine anchor member; the outer shaft including a hook portion formed at the distal end configured to hook the rod for removal of the rod from the spinal anchor member.

2. The rod removal instrument of claim 1 wherein the hook portion forms a rod entryway and a rod receiving portion; both the rod entryway and rod receiving portion are interconnected and sized to receive the rod.

3. The rod removal instrument of claim 1 further including an inner shaft connected to the outer shaft; the inner shaft being located inside the outer shaft and configured to be longitudinally movably with respect to the outer shaft.

4. The rod removal instrument of claim 3 wherein the inner shaft includes a notch formed at the distal end; the inner shaft being connected to the outer shaft such that the notch is displaced a distance from the rod entryway of the outer shaft; the notch being configured to conform to and to partially encompass or cover a rod.

5. The rod removal instrument of claim 3 wherein the distal end of the inner shaft is configured to retain the rod to the instrument when the inner shaft is moved distally to contact the rod.

6. The rod removal instrument of claim 3 wherein the inner shaft is configured to dislodge the rod from the spinal anchor member when the inner shaft is moved distally to contact the rod.

7. A rod removal instrument for removing a rod connected to an anchor member implanted in a patient's spine; the rod removal instrument comprising:
an elongated member having a distal end configured to percutaneously disconnect a rod from an anchor member to which the rod is connected and hold the rod for removal of the rod from the patient.

8. The rod removal instrument of claim 7 wherein the elongated member is an outer shaft.

9. The rod removal instrument of claim 8 further including an inner shaft located within the outer shaft and configured for relative sliding motion with respect to the outer shaft.

10. The rod removal instrument of claim 9 wherein the inner shaft is configured to hold the rod against the outer shaft when the inner shaft is moved against the rod.

11. The rod removal instrument of claim 9 wherein relative sliding motion of the inner and outer shafts acts to dislodge a rod from the bone anchor.

12. The rod removal instrument of claim 7 wherein the distal end is configured to be placed over or cover at least a portion of the anchor member.

13. The rod removal instrument of claim 7 wherein the rod removal instrument is configured for insertion into a patient and removal of a rod from a patient through an opening in a patient that is as large as or slightly larger than the outer perimeter of the distal end.

14. The rod removal instrument of claim 7 wherein the distal end defines a rod entryway interconnected with a rod receiving portion and configured such that the rod is insertable into the rod receiving portion through the rod entryway for removal of the rod from the anchor member.

15. The rod removal instrument of claim 14 wherein the rod removal instrument is configured such that a rod is insertable into the rod receiving portion after passing a portion of the rod through the rod entryway and turning the distal end relative to the rod to insert the rod into the rod receiving portion.

16. A method for removing a rod from a spinal anchor member having a seat implanted in a patient's spine; the method comprising the steps of:
providing a rod removal instrument comprising an outer shaft; the outer shaft having a rod receiving portion at the distal end;
placing the distal end of the rod removal instrument in juxtaposition with the rod to be removed;
hooking the rod into the rod receiving portion;
capturing the rod inside with the rod removal instrument; and
removing the rod from the anchor member.

17. The method of claim 16 further including the steps of:
creating an opening in a patient that is as large as or slightly larger than the outer shaft;
inserting the distal end of the rod removal instrument into the opening; and
removing the rod and rod removal instrument from the opening.

18. The method of claim 16 further including the steps of:
providing a rod removal instrument comprising an inner shaft located within the outer shaft;
moving the inner shaft with respect to the outer shaft such that the rod is captured between the inner shaft and the outer shaft; and
holding the inner shaft against rod.

19. The method of claim 18 wherein the step of moving the inner shaft with respect to the outer shaft includes disengaging any connecting constructs of the rod from the anchor member.

20. The method of claim 16 wherein the step of hooking the rod receiving portion includes turning the outer shaft relative to the rod.
